(19) United States
(12) Patent Application Publication
(10) Pub. No.: US 2008/0233606 A1
(43) Pub. Date: Sep. 25, 2008

(54) USE OF RAMAN SPECTROSCOPY IN ENZYME ACTIVITY ASSAYS

(76) Inventors: Mustapha Haddach, San Diego, CA (US); Gregory S. Naeve, San Francisco, CA (US)

Correspondence Address: WILSON SONSINI GOODRICH & ROSATI, 650 PAGE MILL ROAD, PALO ALTO, CA, 94304-1050, US

(21) Appl. No.: 12/016,915

(22) Filed: Apr. 22, 2008

Related U.S. Application Data
(63) Continuation-in-part of application No. PCT/US06/27486, filed on Jul. 12, 2006.
(60) Provisional application No. 60/700,757, filed on Jul. 18, 2005.

(51) Int. Cl. C12Q 1/26; C12Q 1/00; C07C 321/00; C07C 59/56; C07C 221/00; C07C 233/00; C07C 303/00; C07D 487/12; C07D 263/58; C07D 221/28; C07D 215/44; C07D 311/82

(52) U.S. Cl. 435/25; 435/4; 568/23; 562/472; 564/345; 564/175; 564/42; 540/561; 549/396; 540/2; 548/221; 546/74; 546/160; 549/388

(57) ABSTRACT

Provided herein are assays for detecting enzyme activity using Raman Spectroscopy.

### CROSS-REFERENCE

This application is a continuation-in-part of PCT/US 06/27486 filed on Jul. 12, 2006 and claims the benefit of this application under 35 USC § 365, which claims the benefit of U.S. Provisional Application No. 60/700,757, filed Jul. 18, 2005, each of which are incorporated herein by reference in their entirety.

### SUMMARY OF THE INVENTION

The family of cytochrome P450 (CYP) enzymes are reported to be responsible for the oxidative metabolism of many drugs, pro-carcinogens, pro-mutagens, and environmental pollutants. Cytochrome P450 is a heme-containing, membrane-bound, multi-enzyme system that is present in many tissues in vivo. Cytochrome P450 is generally found at the highest level in the liver. In human liver, it is estimated that there are 15-20 different xenobiotic-metabolizing cytochrome P450 forms. A standard nomenclature based on relatedness of amino acid sequences has been developed. A relatively limited subset of these enzymes, CYP1A2, CYP2C8, CYP2C9, CYP2C19, CYP2D6 and CYP3A4 appear to be most commonly responsible for the metabolism of drugs and associated with drug-drug interactions. See Spatzenegger M. and Jaeger W., Drug Metab. Rev. 27, 397-417, 1995, which is incorporated by reference herein in its entirety.

Identification of the enzymes responsible for metabolism is an important aspect of drug development. Such identification considers both the metabolism of the new drug as well as inhibition by the new drug. The identification of enzymes involved in metabolism of the new drug can also be used to predict how co-administered drug combinations may influence each others metabolism. Furthermore, characterizing a drugs metabolic pathway can also be used to predict individual variability based on known metabolic polymorphisms. Obtaining information for a series of drug candidates early in the drug discovery process can assist in the choice of the best drug candidate for further development.

Current cytochrome P450 assays focus on the metabolism of known organic molecule substrates (see e.g., Table 1) in assessing cytochrome P450 activity and inhibition. While these substrates can be measured by specific assay procedures such as high pressure liquid chromatography (HPLC)/mass spectroscopy or HPLC with radiometry, they are not amenable to high throughput screening assay technology since they require time consuming separation of enzyme reaction products using HPLC. The limited throughput capacity of the current HPLC/mass spectroscopy techniques makes them unsuitable for quickly prioritizing and eliminating the numerous drug candidates identified in the early discovery stages of drug development.

In an attempt to alleviate this bottleneck, high-throughput fluorescence-based methods have been developed as a means to provide a preliminary indication regarding a compound's P450 profile. P450 substrates have been developed specifically to form a fluorescent product to monitor the inhibition of metabolism. However, these compounds are generally not specific for one P450 enzyme and can only be used with individually expressed enzymes, which limits their use to recombinant systems. These substrates cannot be used for in vivo testing of P450 activity. In addition, the inhibitor test molecule and/or its metabolites, if fluorescent, can interfere with the readout of an assay and lead to false negative results. Furthermore, there appears to be a poor correlation with the inhibition profiles obtained using the fluorescent probes relative to those obtained using HPCL/mass spectroscopy methods. See Bjornsson T. H. et al. Drug Metab. Dispos. 2003, 31, 815-832, Stresser et al., Drug Metab. Dispos. 2000, 28, 1440-1448, each of which are incorporated by reference herein in their entirety.

The present invention solves these and other problems by using Raman spectroscopy as the screening method. Due to its high chemical specificity and its use in high throughput analysis, Raman spectroscopy can offer the accuracy and information content available with mass spectroscopy-based methods without the limitations of the high throughput fluorescence-based methods. Thus, Raman spectroscopy-based methods can be used to determine the activity of cytochrome P450 by monitoring the appearance of metabolites that arise from enzyme-specific reactions using probe substrates for each of the cytochrome P450 enzymes.

Described herein is a Raman spectroscopy-based assay useful in the identification of modulators of an enzyme. In one embodiment, the assay is useful to identify inhibitors of an enzyme. In some embodiments, the enzyme is from cytochrome P450 enzymes family.

In certain embodiments, the method comprises the steps of contacting the candidate compound (also referred to herein as the “test compound”), a P450 substrate compound and enzyme under conditions whereby the cytochrome P450 enzyme catalyzes the conversion of the substrate to a cytochrome P450 reaction product (metabolite). After an incubation period, the reaction is stopped, e.g., by adding an acid or solvent and the products of the reaction are extracted from the mixture by an appropriate method. In some, but not all, embodiments, the metabolite is chemically modified with molecules that have strong Raman signals and the susceptibility of the candidate compound to inhibit or activate the enzyme is measured by comparing the Raman spectra of the modified metabolite with Raman spectra of a control reaction (substrate without compound candidate). The change in signal(s) corresponding to the substrate and/or its metabolite is jointly or separately indicative of the activity of the P450 enzyme.

In some embodiments, the assay is useful for identifying potential adverse drug-drug interactions. In still other embodiments, the methods described herein are useful in selecting compounds which inhibit cytochrome P450 enzymes activity. Additionally, the method is useful in selecting compounds which induce cytochrome P450 enzyme activity. Also provided herein are probe substrates useful in a Raman spectroscopy-based assay.

In one embodiment, a method of screening a test compound for its ability to inhibit or induce the cytochrome P450 enzymes is disclosed herein. In some embodiments, the method comprises the steps of incubating the test compound, a cytochrome P450 probe substrate and cytochrome P450 enzyme under conditions whereby the cytochrome P450 enzyme catalyzes the conversion of the probe substrate to a cytochrome P450 reaction product (i.e., metabolite), using conditions generally known to those of ordinary skill in the art. After the incubation period the reaction is stopped and the capability of the test compound to inhibit or induce the appearance of metabolite or metabolites of the P450 substrate is measured by Raman spectroscopy.

The present invention also provides a high throughput method of screening of test compounds for their ability to inhibit or induce the activity of P450 enzymes. In one embodiment, the Raman spectroscopy is performed using SERS-substrates.

Also provided herein are methods for determining the effect of a test compound on the activity of an enzyme, comprising the steps of: (a) combining the test compound with the enzyme and a substrate specific for the enzyme to create a mixture; (b) incubating the mixture under conditions sufficient to promote an enzymatic reaction; (c) subjecting the product from the enzymatic reaction to Raman spectroscopy; and (d) detecting all or part of the signal generated. In some embodiments, the method further comprising the step of (e) comparing the signal generated to a control. In some embodiments, the test compound inhibits the enzyme. In other embodiments, the test compound activates the enzyme.

Also provided herein are methods for determining the effect of a test compound on the activity of a cytochrome P450 enzymes comprising the steps of: (a) combining the test compound with the cytochrome P450 enzyme and a substrate specific for the cytochrome P450 enzyme to create a mixture; (b) incubating the mixture under conditions sufficient to promote an enzymatic reaction; (c) subjecting the product from the enzymatic reaction to Raman spectroscopy; and (d) detecting all or part of the signal generated. In some embodiment, the method further comprises the step of (e) comparing the signal generated to a control. In some embodiments, the assay is performed in a high-throughput fashion.

Also provided herein are methods for determining the effect of a test compound on the activity of an enzyme, comprising the steps of: (a) combining a test compound with an enzyme and a substrate specific for the enzyme to create a mixture; (b) incubating the mixture under conditions sufficient to promote an enzymatic reaction; (c) subjecting the product from the enzymatic reaction to Raman spectroscopy; and (d) detecting all or part of the signal generated from a metabolite of the substrate specific for the enzyme wherein the signal of said metabolite determine the level of enzyme activity. In some embodiments, the methods further comprise the step of (e) analyzing the level of metabolite formed. In other embodiments, the methods further comprise the step of (e) determining the level of the ratio of the substrate to the metabolite. In still other embodiments, the methods further comprise the step of (e) comparing the signal generated to a control.

Also provided herein are methods for determining the effect of a test compound on the activity of an enzyme, comprising the steps of: (a) combining a test compound with an enzyme and a substrate specific for the enzyme to create a mixture; (b) incubating the mixture under conditions sufficient to promote an enzymatic reaction; (c) subjecting the product from the enzymatic reaction to Raman spectroscopy; (d) detecting all or part of a signal generated from a substrate and metabolite and determining the ratio of substrate to metabolite wherein the ratio of said substrate to metabolite indicates a level of enzyme activity. In some embodiments, the method further comprises the step of (e) comparing the signal generated to a control.

Also provided herein are methods for screening one or more test compounds for their effect on an enzyme comprising the steps of: (a) combining the test compound with the enzyme and a substrate specific for the enzyme to create a mixture; (b) incubating the mixture under conditions sufficient to promote an enzymatic reaction; (c) subjecting the product from the enzymatic reaction to Raman spectroscopy; and (d) detecting all or part of the signal generated.

In various embodiments of the methods described herein, the subjecting step comprises using SERS. In some embodiments, SERS is generated using colloidal gold as a SERS-substrate, or by using colloidal silver as a SERS-substrate, or by using coated metal nanoparticles immobilized on magnetic microparticles as a SERS-substrate, or by using coated metal nanoparticles as a SERS-substrate. In other embodiments, the assays described herein are done in a high-throughput fashion.

In various embodiments of the methods described herein, the substrate specific enzyme is selected from the group consisting of midazolam, dixlofenac, testosterone, tolbutamide, felodipine, s-mphenytoin, phenacetin, coumarin, bupropion, amodiaquine, chlorzoxazone, and dextromethorphan.

In some embodiments, the metabolite of the cytochrome P450 substrate is selected from the group consisting of:

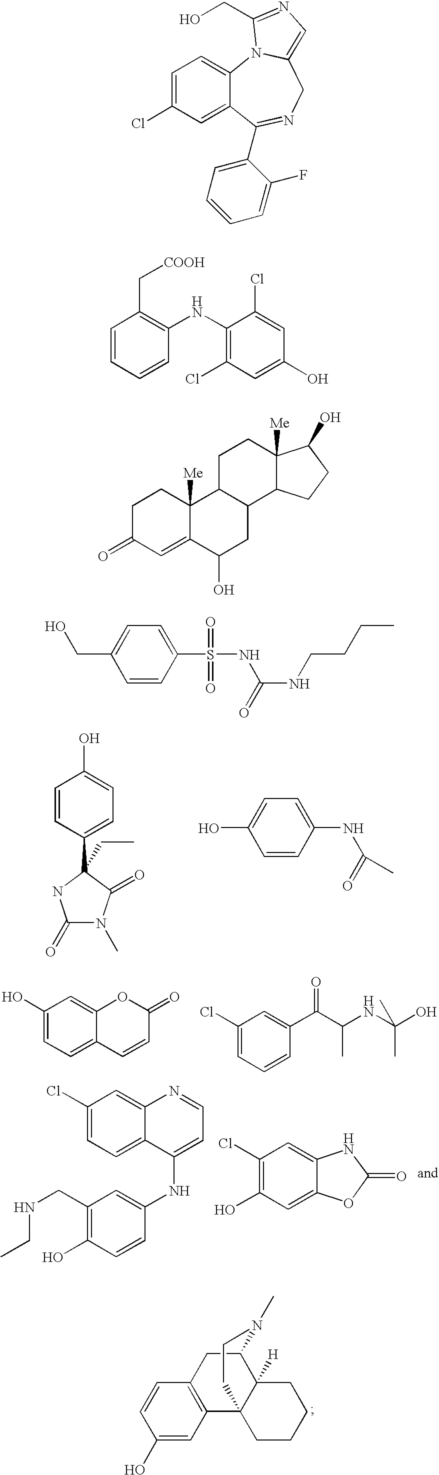

or a deuterated analogue or salt thereof.

In some embodiments, the enzyme used in the methods described herein is a cytochrome P450 enzyme. In various embodiments, the cytochrome P450 enzyme is CYP3A, CYP2E1, CYP2D6, CYP2C19, CYP2C9, CYP2C8, CYP2B6, CYP2A6, CYP1A2.

In other embodiments, the test compound inhibits the enzyme, including but not limited to cytochrome P450. In still other embodiments, the test compound activates the enzyme, including but not limited to cytochrome P450.

In some embodiments, the method described herein further comprises the step of modifying the substrate specific for the enzyme by reacting it with a SERS-active label. In various embodiments, the SERS-active label is a compound of Formula I or Formula II:

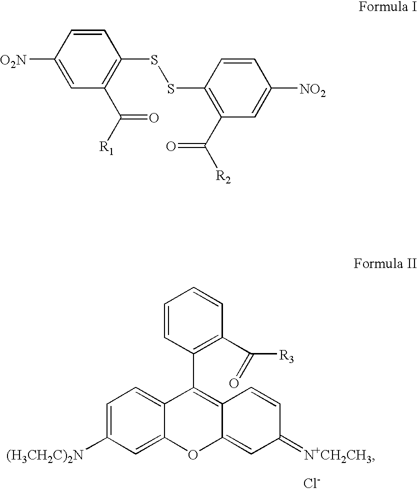

wherein R₁ and R₂ are each independently selected from a hydroxy group or a metabolite of a cytochrome P450 substrate, wherein at least one of R₁ or R₂ is not a hydroxy group; and R₃ is a cytochrome P450 substrate; or a labeled analog, isomer, derivative, or salt thereof. In some embodiments, the metabolite of a cytochrome P450 substrate is selected from the group consisting of:

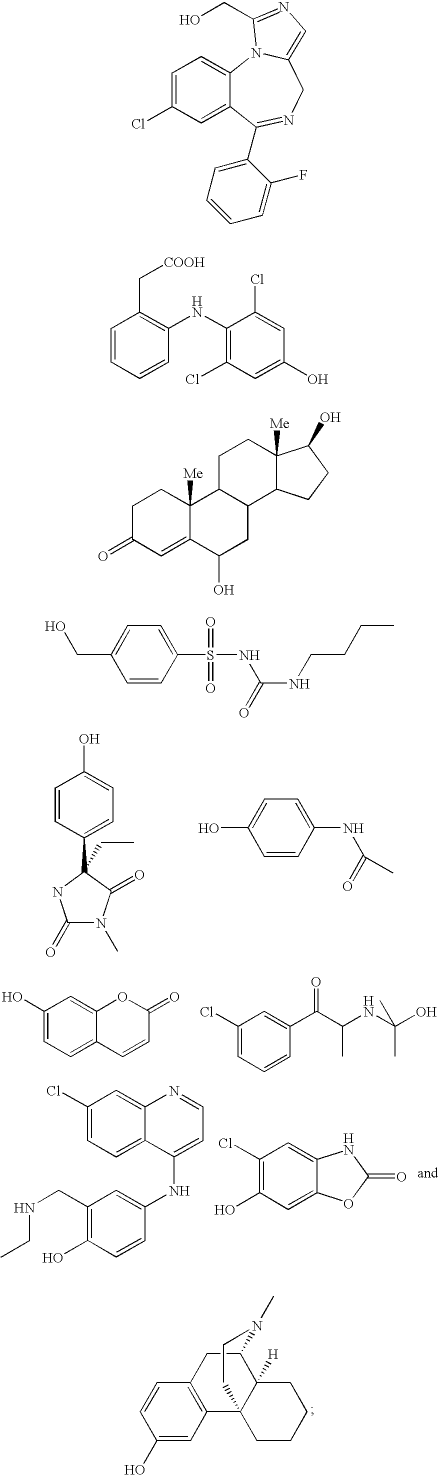

or a deuterated analogue or salt thereof.

Also provided herein are compositions useful in the inventive assays.

Provided herein are compounds of Formula I:

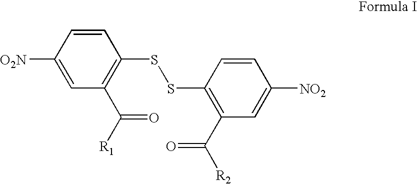

wherein R₁ and R₂ are each independently selected from a hydroxy group or a metabolite of a cytochrome P450 substrate, wherein at least one of R₁ or R₂ is not a hydroxy group; or a labeled analog, isomer, derivative, or salt thereof.

Also provided herein are compounds of Formula II:

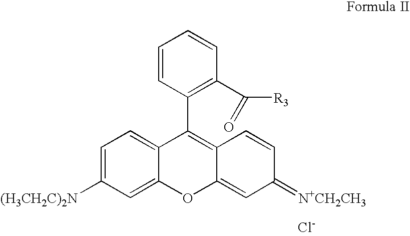

wherein R₃ is a cytochrome P450 substrate, or a labeled analog, isomer, derivative, or salt thereof.

In some embodiments, the metabolite of a cytochrome P450 substrate in Formula I and/or Formula II is selected from the group consisting of:

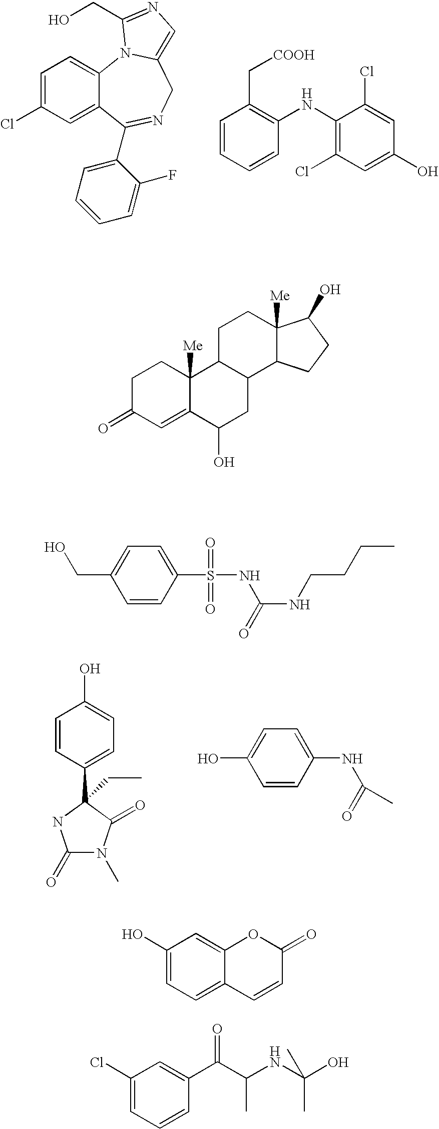

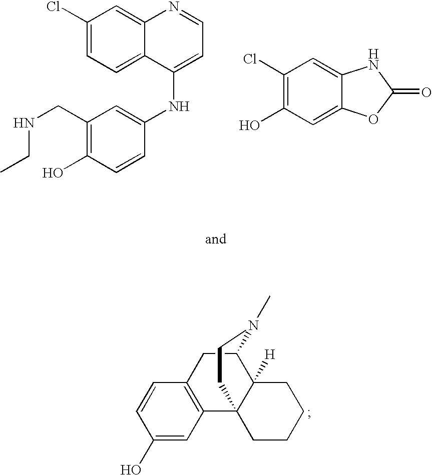

or a deuterated analogue or salt thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the spectra from a SERS-based P450 inhibition assay with Ketonocazole (0.003 μM, 0.03 μM, 0.3 μM and 3.0 μM).

FIG. 2 shows the dose response curve of SERS-based P450 inhibition assay generated by plotting the intensity of NO₂ signal at 1330 cm⁻¹ versus concentration of Ketonocazole.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the claims define the scope of the invention and that methods and structures within the scope of these claims and their equivalents be covered thereby.

The section headings used herein are for organizational purposes only and are not to be construed as limiting the subject matter described. All documents, or portions of documents, cited in the application including, without limitation, patents, patent applications, articles, books, manuals, and treatises are hereby incorporated by reference.

Provided herein are methods for identifying inhibitors or inducers of an enzyme using a Raman spectroscopy-based assay. In one embodiment, the methods are useful in drug discovery for identifying lead compounds. In various embodiments, the enzyme is cytochrome P450. In other embodiments, the assay is useful for identifying potential adverse drug-drug interactions. In still other embodiments, the methods described herein are useful in selecting compounds which inhibit cytochrome P450 enzyme activity. Also provided herein are probe substrates useful in a Raman spectroscopy-based assay, including but not limited to, the Raman spectroscopy-bases assays described herein.

To more readily facilitate an understanding of the invention and its preferred embodiments, the meanings of the terms used herein will become apparent from the context of this specification in view of common usage of various terms and the explicit definitions of other terms provided in the glossary below or in the ensuing description.

Certain Terminology

Unless defined otherwise, all technical and scientific terms used herein have the same meaning as is commonly understood by one of skill in the art to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the internet or other appropriate reference source. Reference thereto evidences the availability and public dissemination of such information.

It is to be understood that the foregoing general description and the following detailed description are exemplary and explanatory only and are not restrictive of any subject matter claimed.

In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include,” “includes,” and “included” is not limiting.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The term “salt” as used herein, refers to salts of the free acids and bases of the specified compound. Compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds of the invention, or by separately reacting a purified compound in its free base form with a suitable organic or inorganic acid, and isolating the salt thus formed. Examples of pharmaceutically acceptable salts include those salts prepared by reaction of the compounds described herein with a mineral or organic acid or an inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate. Other acids, such as oxalic, while not in themselves pharmaceutically acceptable, may be employed in the preparation of salts useful as intermediates in obtaining the compounds of the invention and their pharmaceutically acceptable acid addition salts. (See for example Berge et al., J. Pharm. Sci. 1977, 66, 1-19.) Further, those compounds described herein which may comprise a free acid group may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N⁺(C₁₋₄ alkyl)₄, and the like. Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they may contain. Water or oil-soluble or dispersible products may be obtained by such quaternization. See, for example, Berge et al., supra.

The term “metabolite,” as used herein, refers to a derivative of the compound which is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to the compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

The terms “comprising,” including,” “containing,” and “such as” are used herein in their open, non-limiting sense.

As used herein, the term “test compound” includes any chemical entity such as small organic molecules, peptides and antibodies.

“Enzyme” as used herein is a specific protein which increases (catalyzes) or decreases the speed of a chemical reaction. Example of P450 enzymes include, but are not limited to, CYP3A, CYP2 μl, CYP2D6, CYP2C19, CYP2C9, CYP2C8, CYP2B6, CYP2A6, CYP1A2. Other enzymes include, but are not limited to, lipases, esterases, methyltransferases and proteases.

A “probe substrate” is a molecule which is acted upon by an enzyme. The probe substrate can bind with at least one of the enzyme's active sites which catalyzes a chemical reaction involving the probe substrate. Probe substrates include, but are not limited to, small molecules and peptides.

“Raman spectroscopy” as used herein includes, but is not limited to, SERS, SERRS, resonance Raman spectroscopy, and the like.

The term “SERS-substrate” is any substrate which enhances a Raman signal. Examples of SERS-substrates include, but are not limited to, silver and gold colloids, coated metal nanoparticles, silver or gold colloids immobilized on plastic, silica microspheres on glass slides, silica microspheres on sol-gel films, coated metal nanoparticles immobilized on glass or magnetic microparticles.

The term “coated metal nanoparticles” as used herein are any metal nanoparticles coated with an organic layer such as alkyl groups, aromatic groups, polymers, amino acids, alkyl containing amine groups, alkyl containing acid groups, and the like that can generate SERS.

“Activity” is the chemical reaction that takes place when enzyme is in contact with its probe substrate.

A “SERS-active label” is any molecule that has a strong Raman scatter and can be modified to form a bond, including but not limited to an amide bond or an ester bond, with a substrate of an enzyme.

Certain Chemical Terminology

Definition of standard chemistry terms may be found in reference works, including Carey and Sundberg “ADVANCED ORGANIC CHEMISTRY 4^{TH} ED.” Vols. A (2000) and B (2001), Plenum Press, New York, which is incorporated by reference herein in its entirety. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, —CH₂O— is equivalent to —OCH₂—.

Unless otherwise noted, the use of general chemical terms, such as though not limited to “alkyl,” “amine,” “aryl,” are equivalent to their optionally substituted forms. For example, “alkyl,” as used herein, includes optionally substituted alkyl.

The compounds presented herein may possess one or more stereocenters and each center may exist in the R or S configuration, or combinations thereof. Likewise, the compounds presented herein may possess one or more double bonds and each may exist in the E (trans) or Z (cis) configuration, or combinations thereof. Presentation of one particular stereoisomer, regioisomer, diastereomer, enantiomer or epimer should be understood to include all possible stereoisomers, regioisomers, diastereomers, enantiomers or epimers and mixtures thereof. Thus, the compounds presented herein include all separate configurational stereoisomeric, regioisomeric, diastereomeric, enantiomeric, and epimeric forms as well as the corresponding mixtures thereof. The compounds presented herein include racemic mixtures, in all ratios, of stereoisomeric, regioisomeric, diastereomeric, enantiomeric, and epimeric forms. Techniques for inverting or leaving unchanged a particular stereocenter, and those for resolving mixtures of stereoisomers, or racemic mixtures, are well known in the art and it is well within the ability of one of skill in the art to choose an appropriate method for a particular situation. See, for example, Furniss et al. (eds.), VOGEL'S ENCYCLOPEDIA OF PRACTICAL ORGANIC CHEMISTRY 5^{TH} ED., Longman Scientific and Technical Ltd., Essex, 1991, 809-816; and Heller, Acc. Chem. Res. 1990, 23, 128, each of which are incorporated by reference herein in their entirety.

The compounds presented herein may exist as tautomers. Tautomers are compounds that are interconvertible by migration of a hydrogen atom, accompanied by a switch of a single bond and adjacent double bond. In solutions where tautomerization is possible, a chemical equilibrium of the tautomers will exist. The exact ratio of the tautomers depends on several factors, including temperature, solvent, and pH. Some examples of tautomeric pairs include:

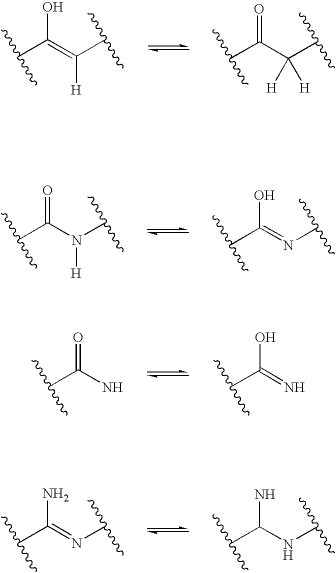

The terms “moiety”, “chemical moiety”, “group” and “chemical group”, as used herein refer to a specific segment or functional group of a molecule. Chemical moieties are often recognized chemical entities embedded in or appended to a molecule.

The term “bond” or “single bond” refers to a chemical bond between two atoms, or two moieties when the atoms joined by the bond are considered to be part of larger substructure.

The term “reactant,” as used herein, refers to a nucleophile or electrophile used to create covalent linkages.

It is to be understood that in instances where two or more radicals are used in succession to define a substituent attached to a structure, the first named radical is considered to be terminal and the last named radical is considered to be attached to the structure in question. Thus, for example, the radical arylalkyl is attached to the structure in question by the alkyl group.

Surface Enhanced Raman Scattering

In various embodiments, the invention described herein provides a technique based on the principle of “surface enhanced Raman scattering” (SERS) and on a modification of that principle known as SERRS (surface enhanced resonance Raman scattering). The principles of Raman scattering are known to skilled artisans and have been used in the detection and analysis of various target materials. Briefly, a Raman spectrum arises because light incident on an analyte is scattered due to excitation of electrons in the analyte. “Raman” scattering occurs when an excited electron returns to an energy level other than that from which it came—this results in a change in wavelength of the scattered light and gives rise to a series of spectral lines at both higher and lower frequencies than that of the incident light. The scattered light can be detected orthogonally to the incident beam.

Normal Raman lines are relatively weak and Raman spectroscopy is therefore too insensitive, relative to other available detection methods, to be of use in chemical analysis. Raman spectroscopy is also unsuccessful for fluorescent materials, for which the broad fluorescence emission bands (also detected orthogonally to the incident light) tend to swamp the weaker Raman emissions. However, a modified form of Raman spectroscopy, based on “surface-enhanced” Raman scattering (SERS), has proved to be more sensitive and hence of more general use. The analyte whose spectrum is being recorded is closely associated with a roughened metal surface. This leads to a large increase in detection sensitivity, the effect being more marked the closer the analyte sits to the “active” surface (the optimum position is in the first molecular layer around the surface, i.e. within about 20 nm of the surface).

The theory of this surface enhancement is not yet fully understood. Without being bound by any particular theory, it is thought that the higher valence electrons of the analyte associate with pools of electrons (known as “plasmons”) in pits on the metal surface. When incident light excites the analyte electrons, the effect is transferred to the plasmons, which are much larger than the electron cloud surrounding the analyte, and this acts to enhance the output signal, often by a factor of more than 10⁶.

A further increase in sensitivity can be obtained by operating at the resonance frequency of the analyte (in this case usually a colored dye attached to the target of interest, although certain target analytes themselves may have suitable color characteristics to use with appropriate lasers). Use of a coherent light source, tuned to the absorbance maximum of the dye, gives rise to a 10³ to 10⁵-fold increase in sensitivity. This is termed “resonance Raman scattering” spectroscopy.

Silver and gold colloids are perhaps the most versatile of substrates used for surface-enhanced Raman spectroscopy (SERS). Aqueous solutions of the colloids are easy to prepare and are stable for long periods of time (Grabar, K. C, et al.; Langmuir 1996, 12, 2353-2361, which is incorporated by reference herein in its entirety). The colloids can be prepared with a wide range of diameters (2.5-120 nm). In some embodiments of the invention described herein, the colloids have an average diameter of about 2 nm, about 3 nm, about 4 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, or about 120 mm. In some embodiments the colloids have an average diameter of about 1 to about 10 nm, or about 5 to about 50 nm, or about 10 to about 100 nm, or about 50 to about 100 nm, or about 75 to 125 nm.

Colloids have been used as a tool to probe the SERS phenomenon. They have been used to examine the roles of surface-active sites and chemical enhancement in SERS (Doering, W. E. et al., J. Phys. Chem. B 2002, 106, 311-317, which is incorporated by reference herein in its entirety) and to evaluate the effects of size and morphology on the magnitude of the SERS effect (Suzuki, M., et al., J. Phys. Chem. B 2004, 108, 11660-11665; Freeman, R. G., et al., J. Raman Spectrosc. 1999, 30, 733-738, each of which are incorporated by reference herein in their entirety). Colloids have also been used to detect bacteria, (Efrima, S., et al., J. Phys. Chem. B1998, 102, 5947-5950; Jarvis, R. M. et al., Anal. Chem. 2004, 76, 40-47. Jarvis, R. M. et al., R. Anal. Chem. 2004, 76, 5198-5202, each of which are incorporated by reference herein in their entirety), nitrogen-containing drugs, (Torres, E. L., et al., Anal. Chem. 1987, 59, 1626-1632, which is incorporated by reference herein in its entirety) and other chemical species (Garrell, R. L. Anal. Chem. 1989, 61, 401A-411A; Angel, S. et al., Appl. Spectrosc. 1990, 44,335-336, each of which are incorporated by reference herein in their entirety). Using running buffers containing silver colloid suspensions, on-column SERS detection in capillary electrophoresis has been demonstrated (Nirode, W. F., et al., Anal. Chem. 2000, 72, 1866-1871, which is incorporated by reference herein in its entirety). Individual colloidal particles have been labeled with reporter molecules and then encapsulated in glass (Mulvaney, S. P. et al., Langmuir 2003, 19, 4784-4790; Doering, W. E., et al., Anal. Chem. 2003, 75, 6171-6176, each of which are incorporated by reference herein in their entirety). The focus of these efforts was to create alternatives to fluorescent tags currently used in genome sequencing, PCR, and immunoassays.

In other efforts, silver/gold colloids have been immobilized on TLC plates (Roth, E., et al., Appl. Spectrosc. 1994, 48, 1193-1195, which is incorporated by reference herein in its entirety), plastic (Supriya, L. et al., Langmuir 2004, 20, 8870-8876), silica microspheres (Fleming, M. S., et al., Langmuir 2001, 17, 4836-4843, which is incorporated by reference herein in its entirety), and on glass slides (Grabar, K. C., et al., J. Anal. Chem., 1995, 67, 735-743, which is incorporated by reference herein in its entirety). They have also been incorporated in sol-gel films to create stable SERS substrates with long shelf lives (Lucht, S., et al., J. Raman Spectrosc., 2000, 31, 1017-1022; Bao, L.; Mahurin, S. M., et al., Anal. Chem. 2004, 76, 4531-4536; Bao, L., et al., Anal. Chem. 2003, 75, 6614-6620, each of which are incorporated by reference herein in their entirety). Immobilized gold colloidal particles on glass have been coated with a C-18 alkylsilane layer and used to detect trace amounts of polycyclic aromatic hydrocarbons (Olson, L. G., et al., Anal. Chem. 2001, 73, 4268-4276, which is incorporated by reference herein in its entirety). Recently pentachlorothiophenol (PCTP)-modified colloidal gold is immobilized on magnetic microparticles and have been used to detect naphthalene by SERS (Boss, P., et al., Anal. Chem., 2005, which is incorporated by reference herein in its entirety). These SERS-substrates are suitable for use in several biological applications since they offer extraction/concentration of the target analyte from a complex sample matrix, ease of separation, suitability for automation, and direct detection using SERS.

High-Throughput Screening of Enzymes

High-throughput screening of thousands of molecules is an important process in drug discovery where it is used to identify compounds that inhibit biological activities and that can therefore serve as lead compounds in medicinal chemistry programs (Cacace A, Drug Discov. Today 8, 785-792, 2003; Khandurina, J, Curr. Opin. Chem. Biol. 6, 359-366, 2002, each of which are incorporated by reference herein in their entirety). More recently, high-throughput screening has become an important technology in basic research laboratories, where it is used to identify small molecules that serve as reagents to study the roles of proteins in cellular processes (Stockwell B. R., Chem. Biol. 6, 71-83, 1999 and Shogren-Knaak M., Annu. Rev. Cell Dev. Biol. 17, 405-433, 2001. Guo Z., Science 288, 2042-2045, 2000, each of which are incorporated by reference herein in their entirety). Many of the assays used in high-throughput screening rely on fluorescent strategies to report on enzymatic activities, including the use of fluorescence resonance energy transfer (FRET) in protease assays (Tawa P. Cell Death Differ. 8, 30-37, 2001, which is incorporated by reference herein in its entirety), fluorescence polarization with labeled antibodies in kinase assays (Fowler A., Anal. Biochem. 308, 223-231, 2002; Parker G. J., J. Biomol. Screen. 5, 77-88, 200, each of which are incorporated by reference herein in their entirety) and environmentally sensitive fluorophores in activity assays (Salisbury C. M., J. Am. Chem. Soc. 124, 14868-14870, 2002, which is incorporated by reference herein in its entirety). The use of a label in these methods can be a detriment, in part because the label can compromise the activity of the probe substrate and in part because some enzymatic activities are not easily adapted to fluorescent labels. In addition, the fluorescence properties of small molecules in the libraries that are tested can lead to false positive signals.

High-Throughput Screening of Enzymes Using Raman Spectroscopy

Recently, high-throughput screening using surface-enhanced resonance Raman scattering (SERRS) has been developed (Barry D Moore, Nature biotechnology, 22, 1133-1138, 2004, which is incorporated by reference herein in its entirety) to screen the relative activities of fourteen enzymes including examples of lipases, esterases and proteases. This approach was made possible by designing “masked” enzyme probe substrates that are initially completely undetected by SERRS. Turnover of the probe substrate by the enzyme leads to the release of surface targeting (silver nanoparticles surface) dye, and intense SERRS signals proportional to enzyme activity. This approach might be applicable to screen for inhibitors of enzymes. However, since it uses a dye label it might suffer from some of the limitations associated with the use of fluorescent labels in high-throughput screening of enzymes.

Cytochrome P450 Assay

In one embodiment, a method of screening a candidate compound for susceptibility to inhibit or activate the P450 enzymes is disclosed herein. In some embodiments, the method comprises the steps of contacting the candidate compound, a P450 probe substrate compound and enzyme under conditions whereby the cytochrome P450 enzyme catalyzes the conversion of the probe substrate to a cytochrome P450 reaction product (metabolite), such conditions are generally known to those of ordinary skill in the art. After an incubation period the reaction is stopped and the ability of the test compound to inhibit or induce the enzyme activity is measured by comparing the Raman spectra of the reaction to Raman spectra of a control reaction (probe substrate without compound candidate). The change in signal(s) corresponding to the probe substrate and/or its metabolite is jointly or separately indicative of the activity of the P450 enzyme.

The present invention also provides a high throughput method of screening of candidate compounds for susceptibility of assaying the activity of cytochrome P450 enzymes.

In Vitro Test Systems

A majority of drugs are cleared via P450-mediated metabolism, therefore the inhibition of P450 enzymes can lead to serious clinical drug interactions. The potential for such interactions is highest when concomitant drugs are metabolized by the same P450 enzyme. In addition, many compounds can also be strong inhibitors of P450 enzymes, which are not directly involved in the clearance of the drug, and could greatly affect the metabolism of co-administered drugs. The information from enzyme inhibition studies is extremely valuable because it could allow extrapolation of the data to other compounds and of drug interactions in organs other than liver (e.g., the intestine) depending upon the degree of the metabolism by the specific organ. The availability of human liver tissue, cDNA expressed P450 enzymes, and specific probe substrates (Table 1) have been valuable tools in the assessment of a drug's potential to inhibit different P450 enzymes in vitro. Inhibition of P450 activity by drugs is most frequently examined in human liver microsomal preparations.

Cytochrome P450 Probe Substrates

In various embodiments described herein, cytochrome P450 probe substrates and their labeled analogues including but not limited to deuterated analogs, are used to determine the inhibition of cytochrome P450 by one or more test compounds using a SERS-based assay. In these embodiments, the metabolite of the probe substrate is detected using a SERS-based assay. In some embodiments, the metabolite is formed and detected in situ.

In some embodiments, the cytochrome P450 substrate is specific for a particular enzyme. In various embodiments, the enzyme the cytochrome P450 substrate is specific for is CYP3A, CYP2E1, CYP2D6, CYP2C19, CYP2C9, CYP2C8, CYP2B6, CYP2A6, or CYP1A2. In some embodiment, the cytochrome P450 substrate is specific for two or more enzymes. In various embodiments, the specificity of the cytochrome P450 substrate for a particular enzyme is at least 2:1, e.g., the specificity of the cytochrome P450 substrate for a particular enzyme can be at least 3:1, 4:1, 5:1, 10:1, 20:1,30:1, 40:1, or 50:1.

In some embodiments, the cytochrome P450 substrate and its metabolite can be detected by SERS. In various embodiments, the ratio of the substrate to its metabolite level is detected and used to determine the activity of a test compound towards the cytochrome P450 enzyme.

In some embodiments, the cytochrome P450 enzyme-specific probe substrate is one of the compounds identified in Table 1 or a labeled analog, derivative or salt thereof.

TABLE 1
Examples of cytochrome P450 probe substrates,
their metabolites and their corresponding
cytochrome P450 enzymes

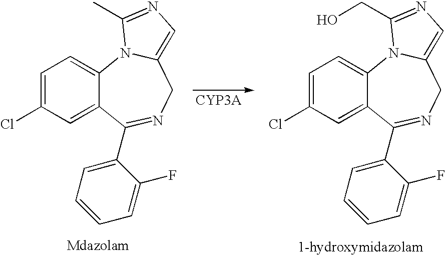

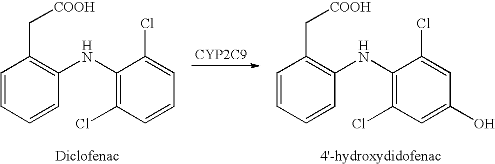

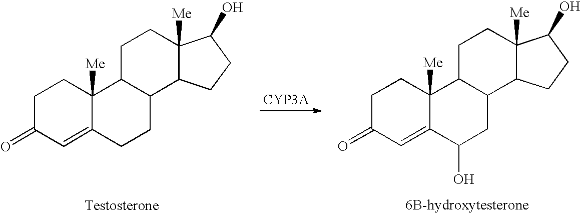

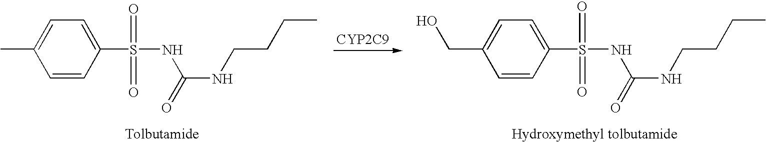

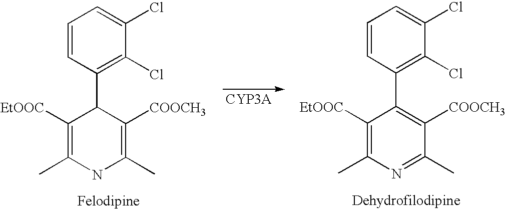

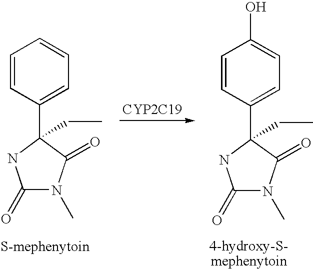

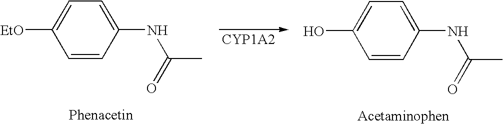

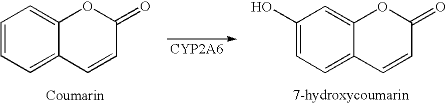

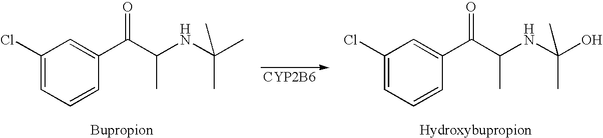

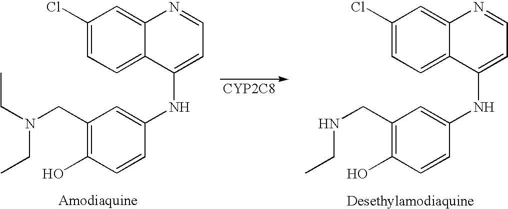

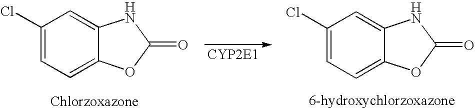

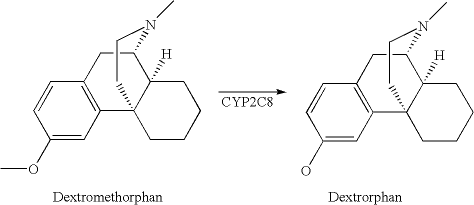

In some embodiments, the probe substrates identified in Table 1, or labeled analogos including but not limited to deuterated analogues, derivatives, isomers or salts thereof, are used to determine specific cytochrome P450 enzyme inhibition by potential drugs.

Chemical Modification of Metabolites

Although in some embodiments of the invention described herein the metabolites are detected by SERS without use of an external label, it other embodiments it is desirable to attach a SERS-active label to the metabolite to produce a strong, characteristic Raman signal that can be easily detected.

SERS-active labels useful in the present invention include, but are not limited to, organic molecules that adsorb well to gold or silver nanoparticle and contain a functional group that can be used to attach the metabolite. In some embodiments of the present invention, the SERS-active label is one described in Table 1 or a derivative thereof.

TABLE 2
Examples of SERS-active labels

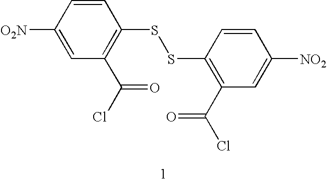

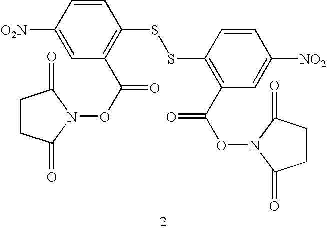

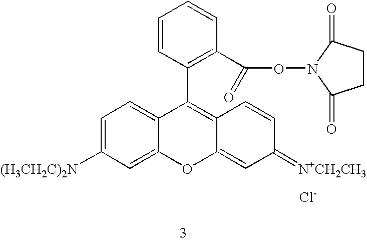

In various embodiments, a metabolite of a cytochrome P450 probe substrate is combined with a SERS-active label and the product is used in the assays described herein to determine the activity of a test compound to cytochrome P450.

In some non-limiting examples, the metabolites described in Table 1 are combined with the SERS-active labels shown in Table 2. In these embodiments, the product is formed by reacting the alcohol or phenol group from the metabolite with the activated site of the SERS-active label to form an ester linkage between the hydroxy group in the metabolite and the activated acid group of the SERS-active label. For example, the metabolite generated from Midazolam by P450 reaction can react with reagent 1 under basic conditions to form the products described in table 3.

TABLE 3
Examples of products of Midazolam metabolite and reagent 1.

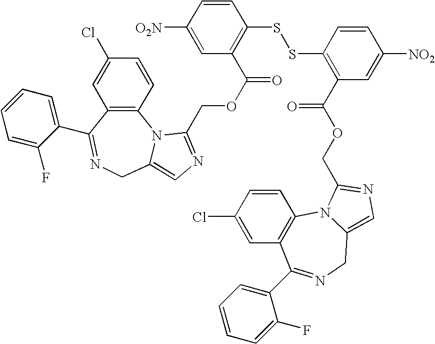

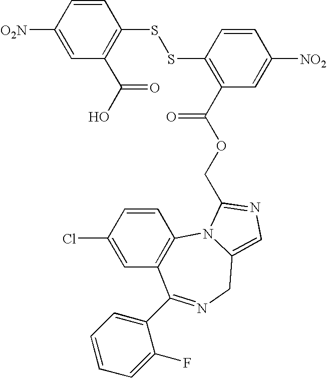

In other embodiments, the product is formed by reacting an amine from a metabolite with the activated site of the SERS-active label to form an amide linkage between the amine in the metabolite and the activated acid group of the SERS-active label. With the teachings provided herein, skilled artisans will recognize additional compounds and linkages useful in the present invention.

In some embodiments, the cytochrome P450 probe substrate is a compound of Formula I:

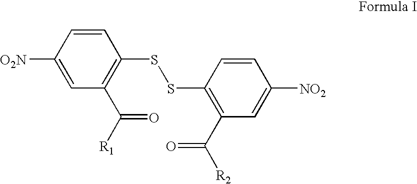

wherein R₁ and R₂ are each independently selected from a hydroxy group or a metabolite of a cytochrome P450 substrate, wherein at least one of R₁ or R₂ is not a hydroxy group. In some embodiments, the metabolite of a cytochrome P450 substrate is selected from the group consisting of:

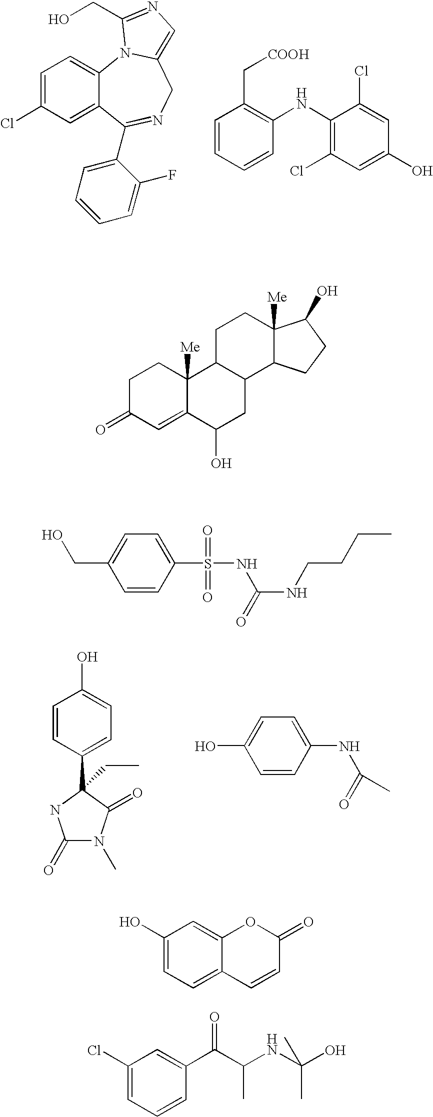

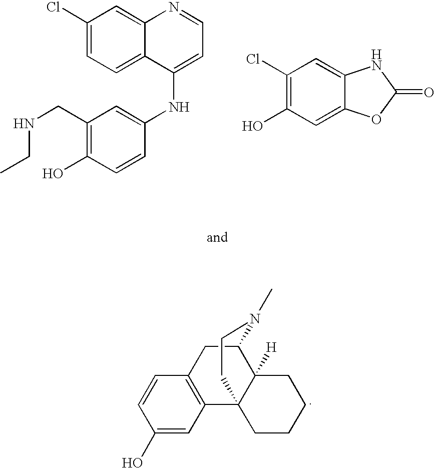

In other embodiments, the cytochrome P450 probe substrate is a compound of Formula II:

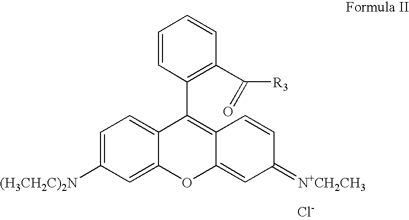

wherein R₃ is a cytochrome P450 substrate, or a labeled analog including but not limited to a deuterated analog, isomer, derivative, or salt thereof. In some embodiments, R₃ is selected from the group consisting of:

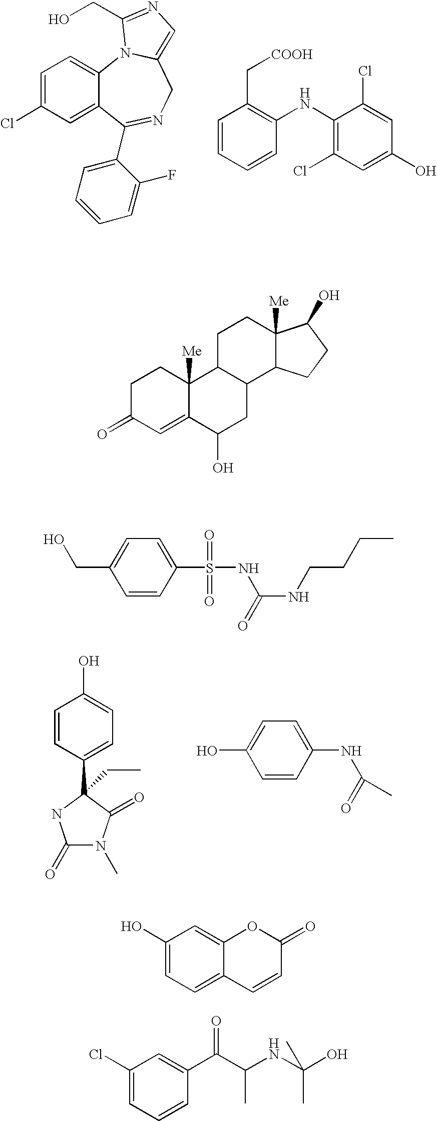

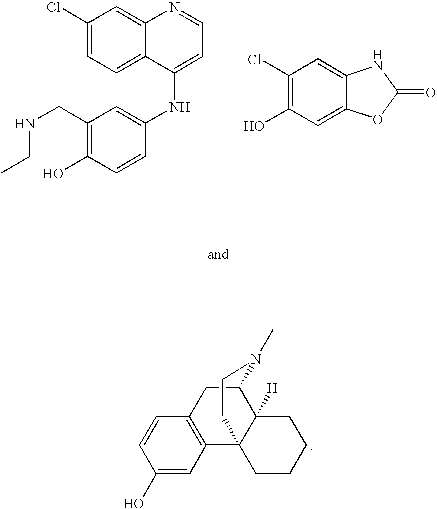

### Synthetic Procedures

Various compounds described and claimed herein can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference in their entirety). General methods for the preparation of compounds as disclosed herein may be derived from known reactions in the field, and the reactions may be modified by the use of appropriate reagents and conditions, as would be recognized by the skilled person, for the introduction of the various moieties found in the formulae as provided herein. As a guide the following synthetic methods may be utilized.

Formation of Covalent Linkages by Reaction of an Electrophile with a Nucleophile

The compounds described and claimed herein can be modified using various electrophiles or nucleophiles to form new functional groups or substituents. The table below entitled “Examples of Covalent Linkages and Precursors Thereof” lists selected examples of covalent linkages and precursor functional groups which yield and can be used as guidance toward the variety of electrophiles and nucleophiles combinations available. Precursor functional groups are shown as electrophilic groups and nucleophilic groups.

| Covalent Linkage Product | Electrophile | Nucleophile |
|---|---|---|
| Carboxamides | Activated esters | Amines/anilines |
| Carboxamides | Acyl azides | Amines/anilines |
| Carboxamides | Acyl halides | Amines/anilines |
| Esters | Acyl halides | Alcohols/phenols |
| Esters | Acyl nitriles | Alcohols/phenols |
| Carboxamides | Acyl nitriles | Amines/anilines |
| Imines | Aldehydes | Amines/anilines |
| Hydrazones | Aldehydes or ketones | Hydrazines |
| Oximes | Aldehydes or ketones | Hydroxylamines |
| Alkyl amines | Alkyl halides | Amines/anilines |
| Esters | Alkyl halides | Carboxylic acids |
| Thioethers | Alkyl halides | Thiols |
| Ethers | Alkyl halides | Alcohols/phenols |
| Thioethers | Alkyl sulfonates | Thiols |
| Esters | Alkyl sulfonates | Carboxylic acids |
| Ethers | Alkyl sulfonates | Alcohols/phenols |
| Esters | Anhydrides | Alcohols/phenols |
| Carboxamides | Anhydrides | Amines/anilines |
| Thiophenols | Aryl halides | Thiols |
| Aryl amines | Aryl halides | Amines |
| Thioethers | Azindines | Thiols |
| Boronate esters | Boronates | Glycols |
| Carboxamides | Carboxylic acids | Amines/anilines |
| Esters | Carboxylic acids | Alcohols |
| Hydrazines | Hydrazides | Carboxylic acids |
| N-acylureas or Anhydrides | Carbodiimides | Carboxylic acids |
| Esters | Diazoalkanes | Carboxylic acids |
| Thioethers | Epoxides | Thiols |
| Thioethers | Haloacetamides | Thiols |
| Ammotriazines | Halotriazines | Amines/anilines |
| Triazinyl ethers | Halotriazines | Alcohols/phenols |
| Amidines | Imido esters | Amines/anilines |
| Ureas | Isocyanates | Amines/anilines |
| Urethanes | Isocyanates | Alcohols/phenols |
| Thioureas | Isothiocyanates | Amines/anilines |
| Thioethers | Maleimides | Thiols |
| Phosphite esters | Phosphoramidites | Alcohols |
| Silyl ethers | Silyl halides | Alcohols |
| Alkyl amines | Sulfonate esters | Amines/anilines |
| Thioethers | Sulfonate esters | Thiols |
| Esters | Sulfonate esters | Carboxylic acids |
| Ethers | Sulfonate esters | Alcohols |
| Sulfonamides | Sulfonyl halides | Amines/anilines |
| Sulfonate esters | Sulfonyl halides | Phenols/alcohols |

Use of Protecting Groups

In the reactions described herein for making compounds useful in the present invention, it may be necessary to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. Protected derivatives are useful in the preparation of the compounds described herein or in themselves may be active as inhibitors. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, or they may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a Pd-catalyzed reaction in the presence of acid labile t-butyl carbamate or base-labile acetate amine protecting groups. Yet another form of protecting group is a resin to which the compound or intermediate may be attached. As long as the residue is attached to the resin, that functional group is blocked and cannot react. Once released from the resin, the functional group is available to react.

Protecting or blocking groups may be selected from:

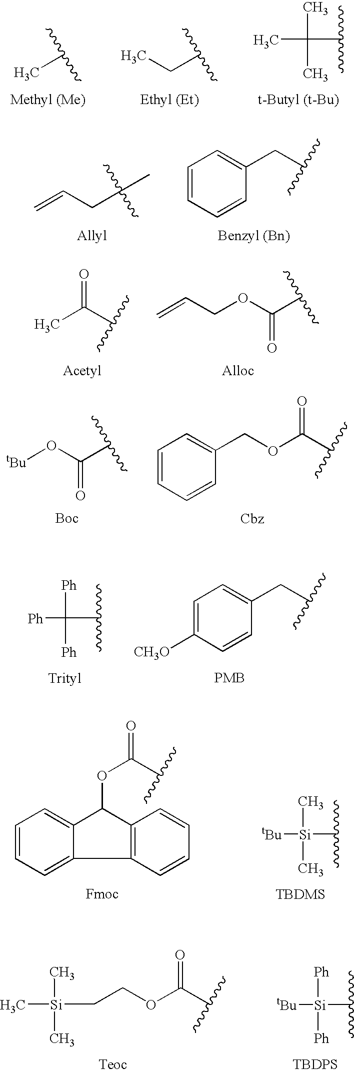

Other protecting groups, plus a detailed description of techniques applicable to the creation of protecting groups and their removal are described in Greene and Wuts, Protective Groups in Organic Synthesis, 3^{rd} Ed., John Wiley & Sons, New York, N.Y., 1999, and Kocienski, Protective Groups, Thieme Verlag, New York, N.Y., 1994, each of which are incorporated herein by reference in their entirety.

Further Forms of the Compounds

Exemplary Isomers

The compounds described herein may exist as geometric isomers. The compounds described herein may possess one or more double bonds. The compounds presented herein include all cis, trans, syn, anti, entgegen (E), and zusammen (Z) isomers as well as the corresponding mixtures thereof. In some situations, compounds may exist as tautomers. The compounds described herein include all possible tautomers within the formulas described herein.

The compounds described herein may possess one or more chiral centers and each center may exist in the R or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion may also be useful for the applications described herein.

In some embodiments, the compounds described herein can be prepared as their individual stereoisomers by reacting a racemic mixture of the compound with an optically active resolving agent to form a pair of diastereoisomeric compounds or complexes, separating the diastereomers and recovering the optically pure enantiomers. While resolution of enantiomers can be carried out using covalent diastereomeric derivatives of the compounds described herein, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The single enantiomer of high optical purity (ee>90%) is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions,” John Wiley And Sons, Inc., 1981, herein incorporated by reference in its entirety.

Exemplary Labeled Compounds

It should be understood that the compounds described herein include their isotopically-labeled equivalents, including their use for treating disorders. For example, the invention provides for methods of treating diseases, by administering isotopically-labeled compounds of formula I. The isotopically-labeled compounds described herein can be administered as pharmaceutical compositions. Thus, the compounds described herein also include their isotopically-labeled isomers, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chloride, such as ²H, ³H, ¹¹C, ¹³C, ¹⁴C, ¹⁵N, ¹⁸ 0, ¹⁷O, ³¹P, ³²P, ³⁵S, ¹⁸F, and ³⁶Cl, respectively. Compounds described herein, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds, pharmaceutically acceptable salts, esters, prodrugs, solvates, hydrates or derivatives thereof can generally be prepared by carrying out procedures described herein, by substituting a readily available isotopically labeled reagent for a non-isotopically labeled reagent.

The compounds described herein may be labeled by other means, including, but not limited to, the use of chromophores or fluorescent moieties, bioluminescent labels, or chemiluminescent labels.

Exemplary Salts

The compounds described herein may also exist as their salts, which can be formed, for example, when an acidic proton present in the parent compound either is replaced by a metal ion, for example an alkali metal ion, an alkaline earth ion, or an aluminum ion; or coordinates with an organic base. Base addition salts can also be prepared by reacting the free acid form of the compounds described herein with an acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

Further, the compounds described herein can be prepared as salts formed by reacting the free base form of the compound with an acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Exemplary Solvates

The compounds described herein may also exist in various solvated forms. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

Exemplary Polymorphs

The compounds described herein may also exist in various polymorphic states. Thus, the compounds described herein include all their crystalline forms, known as polymorphs. Polymorphs include the different crystal packing arrangements of the same elemental composition of the compound. Polymorphs may have different X-ray diffraction patterns, infrared spectra, melting points, density, hardness, crystal shape, optical and electrical properties, stability, solvates and solubility. Various factors such as the recrystallization solvent, rate of crystallization, and storage temperature may cause a single crystal form to dominate.

### EXAMPLES

This invention has been described in an illustrative manner, and it is to be understood that the terminology use is intended to be in the nature of description rather than of limitation. The present invention is further illustrated by the following examples, which should not be construed as limiting in anyway.

### Example 1

### P450 Probe Substrate Inhibition Assays

To determine whether a test compounds inhibits a particular P450 enzyme activity, changes in the metabolism of a cytochrome P450-specific probe substrate (e.g., Table 1) by cytochrome P450 enzyme (e.g., human liver microsomes, or recombinant P450) with varying concentrations of the test compounds are monitored using Raman. A decrease in the formation of the metabolite compared to the vehicle control is used to determine the IC50 value (The concentration at which the metabolism of the P450 probe substrate is reduced by 50%). Known selective P450 inhibitors can be included as control reactions alongside the test compound to assess the validity of the result.

### Example 2

### Cytochrome P450 assay conditions

In general, cytochrome P450 (e.g., human liver microsomes, or recombinant P450) is mixed with phosphate buffer (pH 7.4), MgCl₂, and a P450 specific probe substrate, and warmed to 37° C. in a 96-well plate. Aliquots of this mixture were delivered to each well of a 96-microplate maintained at 37° C. followed by addition of the test compound. Incubations were commenced with the addition of NADPH and maintained at 37° C. Incubations were typically terminated by acidification upon addition of 0.02 ml of termination solvent (e.g. H₂O/CH₃CN/HCOOH; 92:5:3). The terminated reaction mixtures, as well as control samples, composed of the same matrix materials but without test compounds, were passed through a Millipore 96-well filtration apparatus (Millipore Corporation, Billerica, Mass.), containing 0.45-μm mixed cellulose membranes with mild vacuum into a receiver 96-well plate.

The monitoring of the catalytic reaction is performed with SERS-substrates. In general, the substrate and/or metabolic products are extracted using a suitable solid phase extraction matrix (e.g. Waters OASIS ion exchange resins), eluted with a suitable organic solvent (e.g. Methanol) and dried in situ. Extracted compounds are resuspended in a SERS analysis solution containing 20 nm colloidal gold particles, 25 mM KPO₄ buffer, pH 7.2, and 0.1% DMSO. See PCT/US2004/021895, which describes methods for detections substrates using SERS and is hereby incorporated by reference in its entirety.

The Raman spectra signals corresponding to the metabolites and/or probe substrate generated from reactions containing test compounds are compared to control reactions (without test compounds). A decrease in the formation of the metabolite compared to the vehicle control is indicative of an inhibition effect.

### Example 3

### CYP3A4 Inhibition Assay Using Midazolam Probe Substrate

CYP3A4 is the most abundantly expressed constituent in the human liver CYP enzyme system and is also expressed at substantial levels in the intestinal epithelial cells to metabolize orally administered drugs. CYP3A4 is the most important drug metabolizing enzyme, which metabolizes more than 50% of clinical drugs and a wide variety of other xenobiotics, as well as endogenous probe substrates. For example, although beneficial combination therapy utilizing CYP3A4 inhibition has been reported, clinical DDI due to CYP3A4 inhibition often resulted in serious clinical consequences.

A reaction mixture containing a final concentration of 0.04 mg/mL microsomal protein (pooled human liver microsomes) in 0.1 M potassium phosphate buffer (pH 7.4), 5 mM MgCl₂, and 1 mM NADPH in a total volume of 0.5 mL. 5 μM of Midazolam in methanol and varying amounts of test compounds are added into the reaction mixture. The reaction is initiated by the addition of NADPH after a 5 min pre-incubation at 37° C. This experiment is carried out in 96-well plates format in triplicate and includes a control reaction that has no test compound.

### Example 4

### SERS Based Inhibition Assay with Ketonocazole

A 0.2 ml reaction mixture containing 0.5 mg/ml human liver microsomes, 10 mM β-NADPH (20μ), 0.5 M KPO₄ (40 μl, pH 7.4), 30 mM MgCl₂ (12 μl), 1 μl Midazolam (1-80 μM) and 1 μl ketonocazole (1-100 μM) was incubated at 37° C. for 10 min. After incubation the reaction was stopped by the addition of 125 μl acetonitrile and centrifuged (10,000×g) for 3 minutes. The supernatant was isolated and concentrated under vacuum. The residue obtained was partitioned between water and dichloromethane. The organic layer was separated and reagent 1 dissolved in dichloromethane and catalytic amount of dimethyl amino pyridine (DMAP) were added to the organic layer and the mixture was stirred for 30 minutes at room temperature. The mixture was washed several times with 1N sodium hydroxide and concentrated under vacuum. The residue obtained was dissolved in acetonitrile and added to a solution of gold nanoparticles and SERS was measured using 632 nm laser.

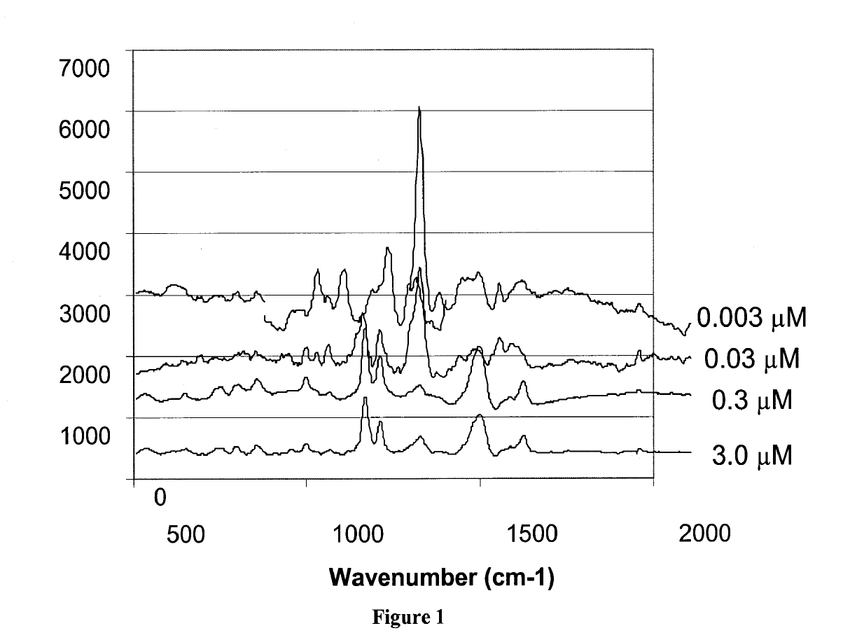

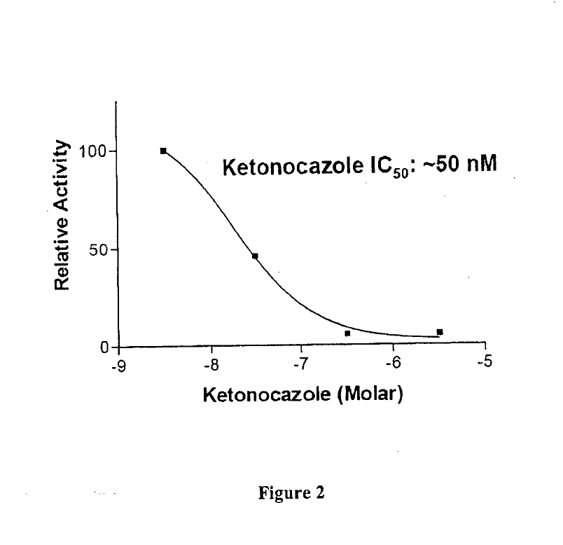

This assay was done with four concentrations of Ketonocazole (0.003 μM, 0.03 μM, 0.3 μM and 3.0 μM) in triplicates. The SERS spectra obtained from the reaction between Midazolame metabolite generated from each concentration and reagent 1 is illustrated in the FIG. 1. The IC₅₀ of Ketonocazole obtained from this experiment was around 50 nM and is shown in FIG. 2.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for determining the effect of a test compound on the activity of an enzyme, comprising the steps of:
(a) combining the test compound with the enzyme and a substrate specific for the enzyme to create a mixture;
(b) incubating the mixture under conditions sufficient to promote an enzymatic reaction;
(c) subjecting the product from the enzymatic reaction to Raman spectroscopy; and
(d) detecting all or part of the signal generated.

2. The method of claim 1, further comprising the step of (e) comparing the signal generated to a control.

3. The method of claim 1, wherein the signal of said metabolite is used to determine the level of enzyme activity.

4. The method of claim 1, further comprising the step of analyzing the level of metabolite formed wherein the higher the metabolite signal, the lower the potency of the test compound.

5. The method of claim 1, further comprising the step of determining the ratio of the substrate to the metabolite.

6. The method of claim 1, wherein SERS is used in the subjecting step (c).

7. The method of claim 6, wherein the SERS is generated using colloidal gold as a SERS-substrate.

8. The method of claim 1, wherein the substrate specific enzyme is selected from the group consisting of midazolam, dixlofenac, testosterone, tolbutamide, felodipine, s-mphenytoin, phenacetin, coumarin, bupropion, amodiaquine, chlorzoxazone, and dextromethorphan.

9. The method of claim 1, wherein the enzyme is a cytochrome P450 enzyme.

10. The method of claim 9, wherein the cytochrome P450 enzyme is CYP3A, CYP2E1, CYP2D6, CYP2C19, CYP2C9, CYP2C8, CYP2B6, CYP2A6, CYP1A2.

11. The method of claim 9, wherein the metabolite of the cytochrome P450 substrate is selected from the group consisting of: 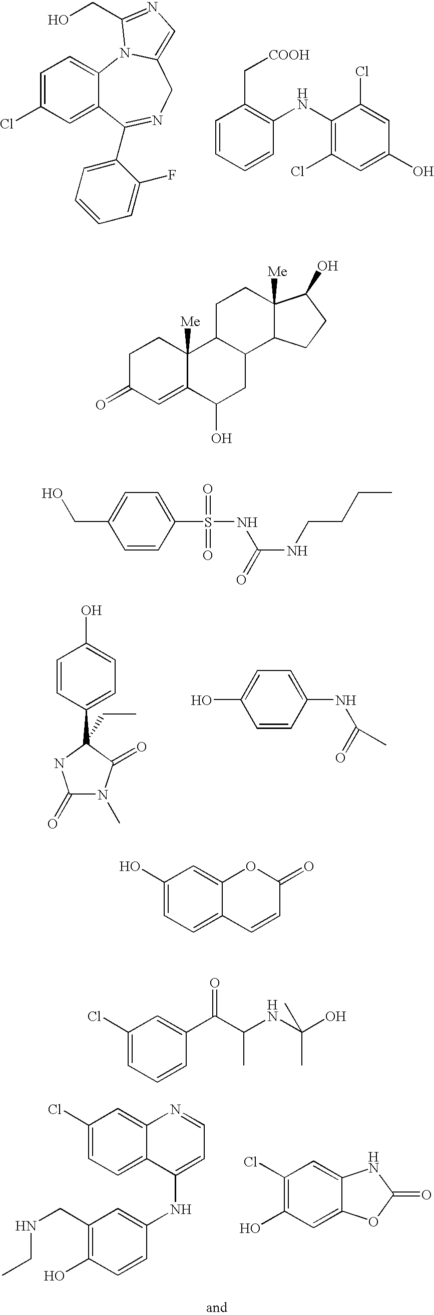 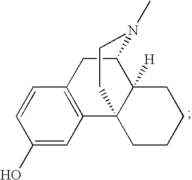
or a deuterated analogue or salt thereof.

12. The method of claim 1, wherein in step (a) the test compound is combined with a cytochrome P450 enzyme to create a mixture and in step (c) SERS is used.

13. The method of claim 1 or 13, further comprising a step of modifying the substrate specific for the enzyme by reacting it with a SERS-active label.

14. The method of claim 13, wherein the SERS-active label is a compound of Formula I or Formula II: 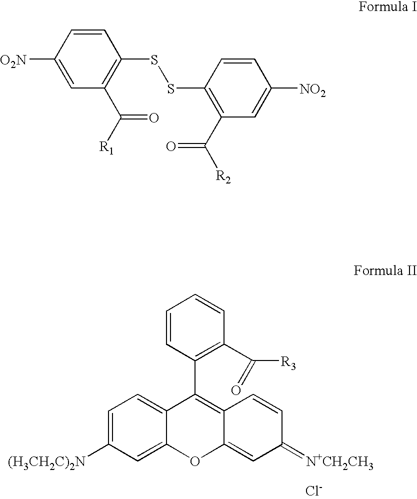
wherein:
R₁ and R₂ are each independently selected from a hydroxy group or a metabolite of a cytochrome P450 substrate, wherein at least one of R₁ or R₂ is not a hydroxy group; and
R₃ is a cytochrome P450 substrate; or a labeled analog, isomer, derivative, or salt thereof.

15. The method of claim 14, wherein the metabolite of a cytochrome P450 substrate is selected from the group consisting of: 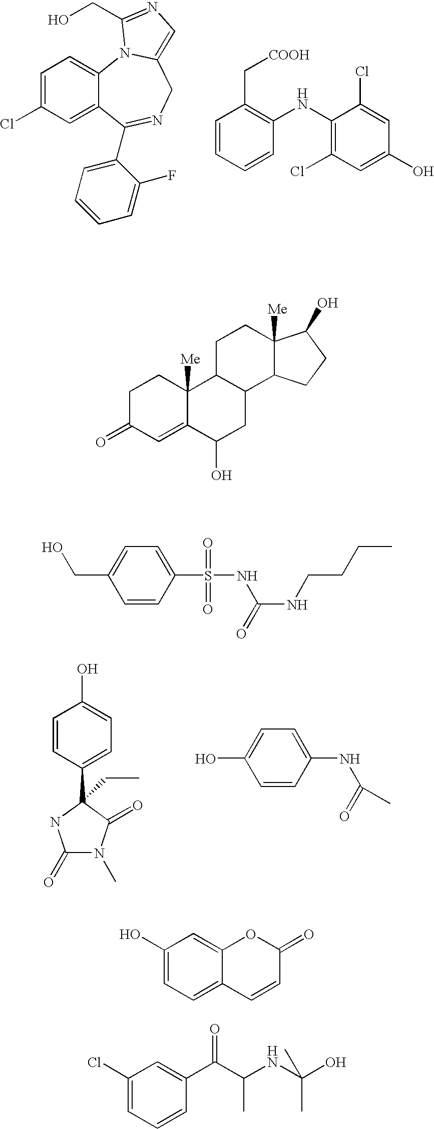 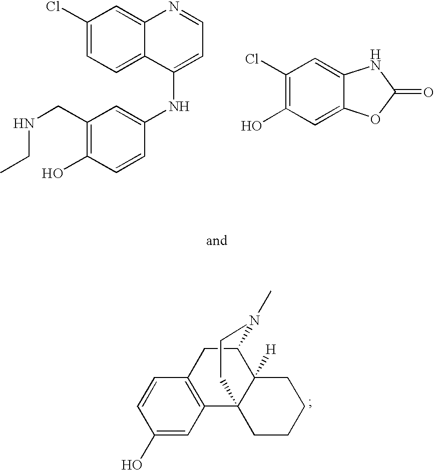
or a deuterated analogue or salt thereof.

16. A compound of Formula I: 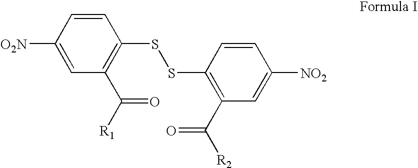
wherein R₁ and R₂ are each independently selected from a hydroxy group or a metabolite of a cytochrome P450 substrate, wherein at least one of R₁ or R₂ is not a hydroxy group;
or a labeled analog, isomer, derivative, or salt thereof.

17. A compound according to claim 16, wherein the metabolite of a cytochrome P450 substrate is selected from the group consisting of: 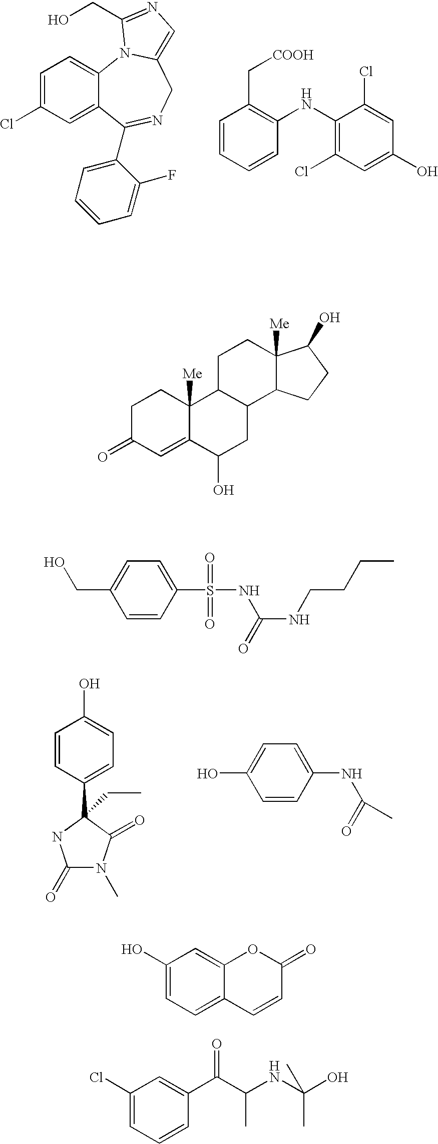 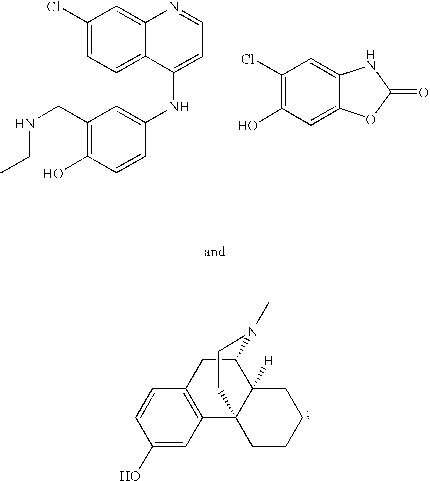
or a deuterated analogue or salt thereof.

18. A compound of Formula II: 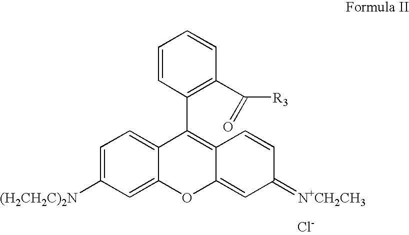
wherein R₃ is a cytochrome P450 substrate, or a labeled analog, isomer, derivative, or salt thereof.

19. A compound according to claim 18, wherein the metabolite of a cytochrome P450 substrate is selected from the group consisting of: 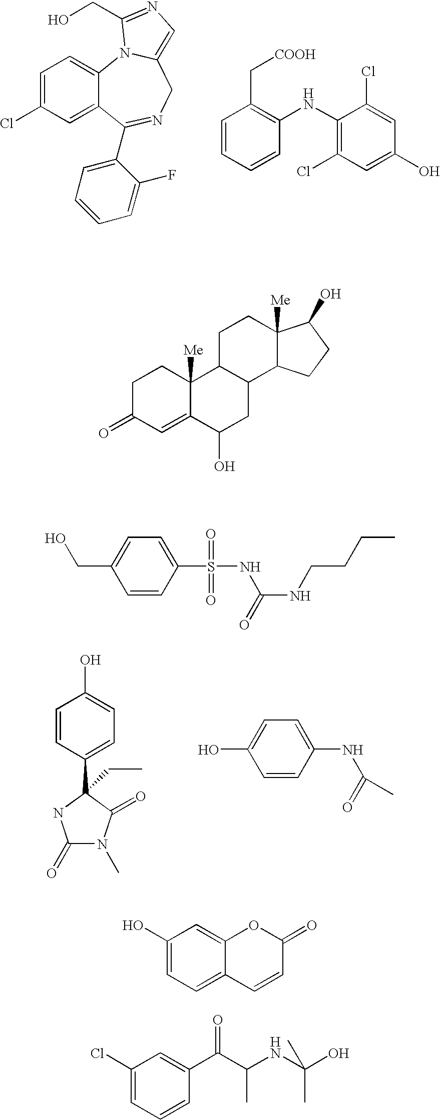 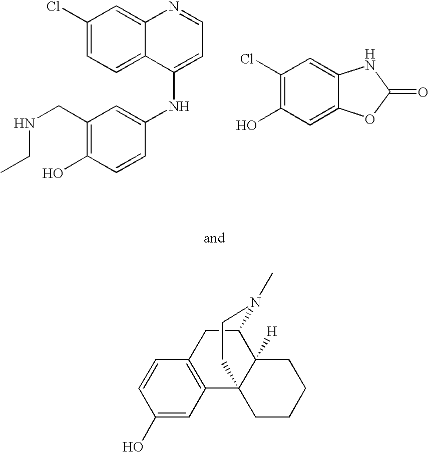
or a deuterated analogue or salt thereof.
